(19) United States
(12) Patent Application Publication
(10) Pub. No.: US 2004/0242676 A1
(43) Pub. Date: Dec. 2, 2004

(54) Method of mitigating the adverse effects of IL-2

(76) Inventors: Thomas R. Alessi, Hayward, CA (US); S. Mark Moran, Orinda (CA)

Correspondence Address: COOLEY GODWARD, LLP, 3000 EL CAMINO REAL, 5 PALO ALTO SQUARE, PALO ALTO, CA, 94306, US

(21) Appl. No.: 10/452,324

(22) Filed: May 30, 2003

(51) Int. Cl. A61K 31/366

(52) U.S. Cl. 514/457; 514/469

(57) ABSTRACT

A new polymorphic form of (±)7-[3-(4-acetyl-3-methoxy-2-propylphenoxy)propoxy]-3,4-dihydro-8-propyl-2H-1-benzopyran-2-carboxylic acid is disclosed. The compound has a melting point of 80° C. to 82° C. and is a leukotriene B4 antagonist (“LBA”). The compound is useful for diminishing the adverse effects (e.g. vascular leakage syndrome) of IL-2 treatment.

Administering the compound with IL-2 diminishes the adverse effects of IL-2 and preserves or enhances the beneficial effects of LBA administration on a subject, while simultaneously mitigating the adverse effects of using a LBA. The method involves administering an amount of LBA, preferably the new polymorphic form, to a subject undergoing IL-2 treatment, where the amount administered is such that the LBA is maintained in a specified range over the treatment schedule. Also disclosed is an article of manufacture comprising a composition of the new polymorph with labeling instructions for treatment. Also disclosed is a method for preparing a pharmaceutical composition of the new polymorph and a method for preparing the polymorph itself.

### TECHNICAL FIELD

This invention relates to a hypotoxic polymorphic form of a leukotriene B₄ receptor antagonist, that exhibits an improved therapeutic index. This invention also relates to an improvement the therapeutic index of other drugs (such as interleukin-2) that induce leukotriene-mediated adverse side effects.

### BACKGROUND

Recombinant interleukin-2 (PROLEUKIN® or “IL-2”) is an analogue of human native interleukin-2. While human native interleukin-2 is present in a human in small amounts, under certain conditions, such as the administration of IL-2 to treat certain conditions, excessive levels (i.e., higher-than-normal levels) of IL-2 will be present in a subject's system. IL-2 is approved for the treatment of certain human malignancies including melanoma and renal cell carcinoma and is also useful for the treatment of certain viral conditions. The administration of IL-2 has been associated with “vascular leak syndrome” (VLS), which results from extravasation of plasma proteins and fluid from the vasculature into the extravascular space. It is known that, among other adverse signs or symptoms, VLS can cause generalized edema, systemic hypotension, reduced organ perfusion, and subsequent dysfunction of one or more tissues or organs. When sufficiently severe, VLS may cause significant disability or even death. The adverse effects of IL-2 may necessitate withholding doses or using a lower dose of IL-2, thereby diminishing the potential for therapeutic benefit from IL-2. An effective means of mitigating IL-2 adverse effects would be beneficial.

However, a pharmacological intervention that mitigates the adverse effects of any drug such as IL-2 may also mitigate the beneficial effects. Until the discovery set forth in U.S. Pat. No. 6,423,744 B1, issued 23 Jul. 2002, it was a deficiency of prior art that no intervention to prevent or mitigate VLS had been shown also not to interfere with or, more preferably, to enhance the antitumor activity of interleukin-2. In that patent, it was demonstrated that IL-2 increases plasma levels of leukotrienes, including leukotriene B₄ and that using a leukotriene B₄ (LTB₄) receptor antagonist during IL-2 therapy mitigates VLS, lessens the adverse effects of IL-2, without interfering with the beneficial antitumor effects of IL-2.

A particular LTB₄ receptor antagonist (“LBA”), (±)-7-[3-(4-acetyl-3-methoxy-2-propylphenoxy)propoxy]-3,4-dihydro-8-propyl-2H-1-benzopyran-2-carboxylic acid, was found to be useful in this role.

Specific blockade of the leukotriene B4 receptor may not only mitigate VLS and lessen the adverse effects of IL-2 but can also preserve or enhance antitumor activity, thereby improving the therapeutic index of IL-2.

Therapeutically beneficial modulation of cytokine pathways requires control of the level of modulation. In the setting of IL-2 administration and the generation of LTB₄, there are three possible outcomes of attempted blockade of the LTB₄ receptor:

1. inadequate blockade, which might lead to persistence or recurrence of adverse effects from IL-2

2. excessive blockade, which might lead to unexpected and undesirable adverse side effects

3. appropriate, well modulated blockade that produces the desired improvement in therapeutic index of IL-2.

It is well known in the art that the dose of a drug does not always correlate with blood or tissue levels. Drug levels for a given dose may vary widely as a function of many different parameters such as the age, sex, weight, diet, the presence or absence of food before/during/after drug administration, species of animal receiving the drug, concomitant medical or surgical treatments, multiple medical or surgical abnormalities, and the like. Therefore, to achieve the safe and effective use of many drugs, precise knowledge of such blood or tissue levels is required. It is a failing of prior art that no guidance has been provided regarding appropriate blood or tissue levels of any drug, such as a LBA or other IL-2 modulating agent, to effect an improvement of the therapeutic index of IL-2. Therefore, specific knowledge of the required blood or tissue levels of an agent that may affect the therapeutic index of IL-2 would be of particular benefit, and the availability of an appropriate assay or kit, would be of particular benefit. In the absence of such information, it is a further failing of prior art that there is no guidance regarding appropriate blood or tissue levels of any drug, such as a LBA or other IL-2 modulating agent, to effect an improvement of the therapeutic index of IL-2.

However, treatments designed to reduce the serious adverse side effects of another therapy can in and of themselves manifest toxicity although usually of a different and milder form. To optimize overall therapy, then, it is desirable not only to reduce or eliminate the toxicity of the primary therapy (e.g., interleukin-2) via concomitant administration of a mitigating agent (i.e., an LBA) but also to reduce or eliminate any toxicity associated with the mitigating agent itself.

It is known in the art that in certain instances a single compound may crystallize into two or more structurally distinct forms, i.e. polymorphs. Differences in crystalline structure can be associated with varying physicochemical characteristics of the various polymorphs. Such variation can also be the source of difficulties or inefficiencies in the manufacturing of a particular molecular structure. These difficulties or inefficiencies may include, by way of examples, alterations in the yield of a manufacturing process or the need to develop and adopt modified analytical methodology. In the setting of manufacturing under the standards of Good Manufacturing Practices (as described in the United States Code of Federal Regulations and related laws and guidelines), changes in manufacturing specifications may be a direct result of the generation of differing polymorphic forms during manufacturing. Such differences in specifications can result in losses in time and increases in manufacturing costs. While it is known in the art that polymorphism can occur, when it will occur is unpredictable. Whether such occurrence is beneficial, detrimental, or neutral in effect is also unpredictable.

It is generally believed that the characteristics of a molecule in solution (and therefore in the absence of any crystalline structure) are independent of the particular polymorphic form, if any, from which the molecule was derived. However, because of thermodynamic polymorphic variation, there may be mandatory conditions to achieve solubilization at all. These may be, for example, differences in pH, temperature, time, or the need for co-solvents in order to achieve dissolution. There may also be important interactions between variables such as these to effect dissolution. By way of example, one polymorphic form may dissolve optimally at a given pH for a given period of time (pH-time interaction) while a second polymorphic form may dissolve better at a different pH or within a different time (a second pH-time interaction). Since local pH varies widely in the body, and residence time for molecules within tissues can also vary widely for different drugs or different structures, such interactions can have important biological consequences in the treatment of humans with particular polymorphic forms.

With these considerations in mind, it is appropriate to note that many drugs are administered orally and traverse the various segments of the gastrointestinal tract during the process of dissolution and absorption. The pH of the gastrointestinal tract varies from quite acidic to quite basic according to anatomic location. The stomach(s) in most species tend to have an acid pH while more distal segments of the bowel (duodenum or jejunum) are more or frankly alkaline in pH. The time required for a molecule to traverse a particular segment of the gastrointestinal system, e.g., stomach or small bowel, can also vary as a function of pH, the presence of foods, other diseases, and the like. In addition, it is also possible that significant differences in metabolism or pharmacokinetics (and thereby related pharmacodynamics) may occur as a function of where in the gastrointestinal tract a molecule is first dissolved and absorbed. We have discovered that such differences can have important implications for the choice of a polymorphic form for use in treatment.

We have now discovered that when a LBA is used during the administration of IL-2 for treatment of certain human malignancies and human viral conditions, the plasma level of the LBA must be maintained within a carefully defined range. Such maintenance can improve the therapeutic index of IL-2. Furthermore, by maintaining the LBA within the desired range, both the dose level of IL-2 can be increased and the length of treatment can be extended. This results in a therapeutic index that is greater than seen previously, i.e. the adverse effects of IL-2 treatment are reduced more than a reduction in anti-tumor or anti-viral effects.

We have also discovered a new polymorphic form of the specific LBA mentioned above that has surprisingly low toxicity and provides a significant advantage over the general teaching of the prior art. The use of this specific polymorphic form of the compound identified above as concomitant therapy to IL-2 results in both a reduction in IL-2 induced adverse events while also mitigating or eliminating any significant adverse events associated with the LBA itself. With such a dual mitigation strategy, and the careful maintenance of the plasma level of the LBA being used in the subject, the beneficial effects for patients and medical care listed above will be increased even more, thereby leading to an even better tolerated treatment, a further reduction in the cost of treatment, and an even better response thereto.

### SUMMARY OF THE INVENTION

One aspect of this invention is a method for treating a malignancy, viral disease, or immunologic disease in a subject having such a condition. The method comprises

(a) administering leukotriene B₄ antagonist (LBA) to the subject to maintain a level of the LBA in the subject's plasma within a target range,

(b) concurrently administering IL-2 at a level that is the same or greater than the dosage that can be tolerated by the subject for IL-2 administered in the absence of the LBA,

(c) continuing to administer LBA and IL-2 to maintain the LBA within the individual target range, and

(d) optionally increasing the dosage of either or both the LBA and IL-2 if, after monitoring the subject's vital signs and/or laboratory parameters, such increase is warranted.

Another aspect of this invention is the compound of (O)-7-[3-(4-acetyl-3-methoxy-2-propylphenoxy)propoxy]-3,4-dihydro-8-propyl-2H-1-benzopyran-2-carboxylic acid, which exhibits a melting point of about 80° C.-82° C. This compound is particularly useful in the method for treatment described herein.

Another aspect of this invention is a method for mitigating the adverse effects of the administration of IL-2 to a subject undergoing IL-2 treatment for a malignancy, a viral disease, or an immunologic disease, which method comprises administering the compound, as described above, to the human in an amount and for a time sufficient to improve the therapeutic ratio of the IL-2.

Another aspect of this invention is a pharmaceutical composition that comprises the compound described above in combination with a pharmaceutically acceptable excipient.

Still another aspect of this invention is an article of manufacture that comprises the pharmaceutical composition described above in a container associated with printed instructions for administering the pharmaceutical composition to a human subject having an IL-2 treatable malignancy, viral diseases, or immunologic disease in conjunction with IL-2 to treat such malignancy or infection. This improves the therapeutic index of IL-2.

Still another aspect of this invention is a process for preparing a pharmaceutical composition, which process comprises combining the compound described above with a pharmaceutically acceptable excipient.

Still a further aspect of this invention is a process for preparing the compound described above, which process comprises dissolving a starting material of (O)-7-[3-(4-acetyl-3-methoxy-2-propylphenoxy)propoxy]-3,4-dihydro-8-propyl-2H-1-benzopyran-2-carboxylic acid in ethyl acetate, cooling the resulting solution below 10° C., adding hexane to the solution while mixing until a precipitate forms, separating the precipitate from the liquid, and drying the precipitated material.

Another aspect of this invention is a method for assaying the effectiveness of treatment of a patient having a malignancy, a viral disease, or immunological disease, with IL-2 in conjunction with a leukotriene B₄ antagonist (“LBA”), which method comprises monitoring the patient's plasma levels for the LBA to determine the concentration of the LBA and adjusting the amount of the LBA administered to the patient to ensure the LBA concentration is maintained in a fixed range.

Another aspect of the invention can be viewed as a method for mitigating the adverse effects of the administration of IL-2 to a human undergoing IL-2 treatment for a malignancy, a viral disease, or immunological disease. The method comprises administering the compound of the invention to the human in an amount and for a time sufficient to improve the therapeutic ratio of the IL-2.

Another aspect of the invention is an improvement in a method of treating a subject having a malignancy, a viral disease, or an immunological disease with IL-2 in conjunction with a leukotriene B₄ receptor agonist (“LBA”). The improvement comprises maintaining the subject's plasma level of the LBA within a target range during the IL-2 treatment.

Another aspect is the mitigation of LBA-related adverse events by the use of a specific polymorphic form of the LBA. In a process for treating a malignancy, a viral disease, or immunological disease using IL-2 in combination with a LBA such as (±)-7-[3-(4-acetyl-3-methoxy-2-propylphenoxy)propoxy]3,4-dihydro-8-propyl-2H-1-benzopyran-2-carboxylic acid, the improvement comprises mitigating the LBA-related adverse events by using a specific polymorphic form of the LBA such as the compound named above having a melting point of 80° C. to 82° C.

Another aspect of this invention is the determination of a target range for the LBA on a patient specific basis.

Other aspects of the invention will be apparent to one of skill in the art upon reading the ensuing detailed description of the invention.

### DESCRIPTION OF THE FIGURES

FIG. 1: This figure presents comparative in vivo results showing the effects of the compound useful in this invention on the prevention of reduced oxygenation of arterial blood by IL-2 administration.

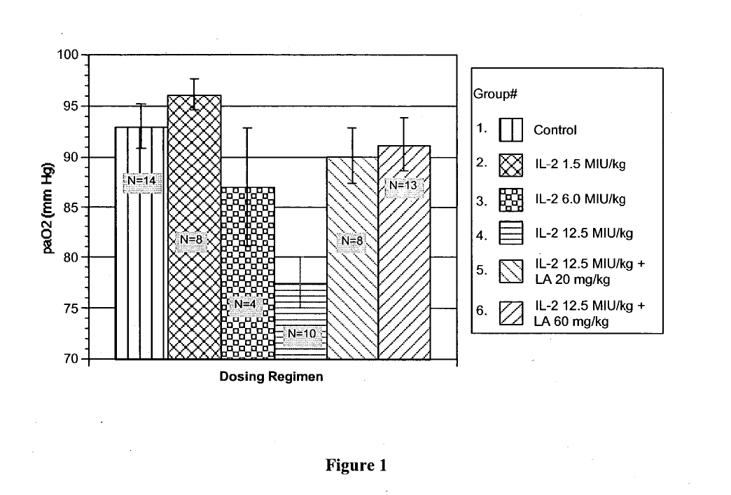

FIG. 2: This figure presents the number of doses of IL-2 that could be safely administered to patients with metastatic renal cell cancer as related to the dose of the LBA compound useful in this invention. As LBA plasma level rises, the number of well-tolerated doses of IL-2 also increases (p<0.05).

FIG. 3: This figure presents the number of doses of IL-2 that could be safely administered to patients with metastatic renal cell cancer as related to the plasma level of the LBA compound useful in this invention. As LBA plasma level rises, the number of well-tolerated doses of IL-2 also increases (p<0.03).

FIG. 4: This figure presents the number of clinically serious adverse events caused by exposure to IL-2 as related to the dose of the leukotriene B4 antagonist useful in this invention. As the LBA dose rises, the number of well-tolerated doses of IL-2 also increases.

FIG. 5: This figure provides an infrared spectrum of the high melting point polymorph of this invention.

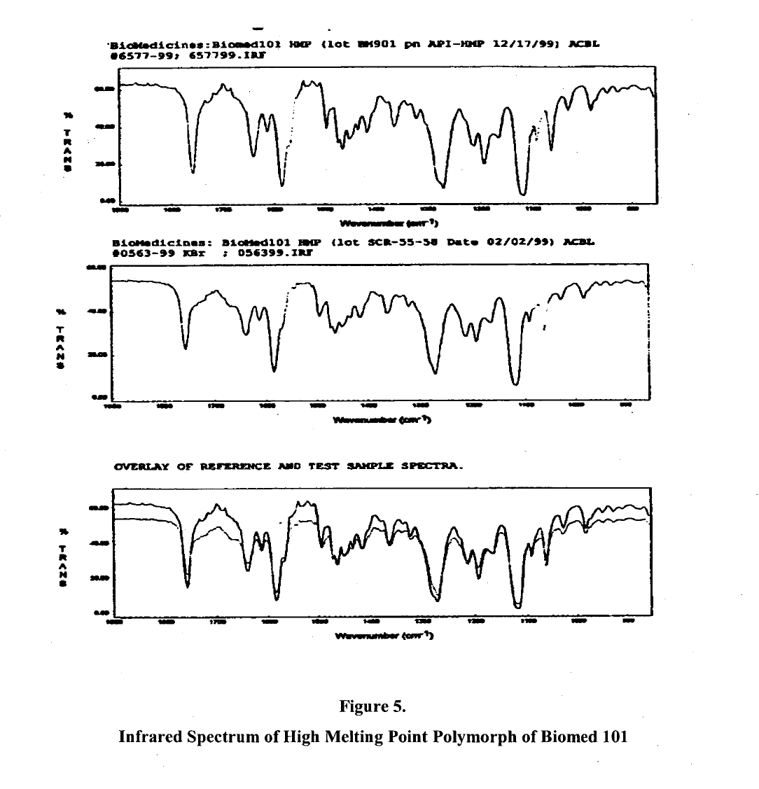

FIG. 6: This figure provides comparative infrared spectra of the high melting point compound of this invention along with the known low melting point compound.

FIG. 7: This figure provides 12 month stability data for the compound of this invention formulated in 25 mg capsules.

FIG. 8: This figure provides 6 month and 12 month stability data for the compound of this invention formulated in 50 mg capsules.

FIG. 9: This figure provides a photomicrograph of the known, low melting point polymorphic form of (±)-7-[3-(4-acetyl-3-methoxy-2-propylphenoxy)-propoxy]3,4-dihydro-8-propyl-2H-1-benzopyran-2-carboxylic acid. This is designated as BMED 101 LMP.

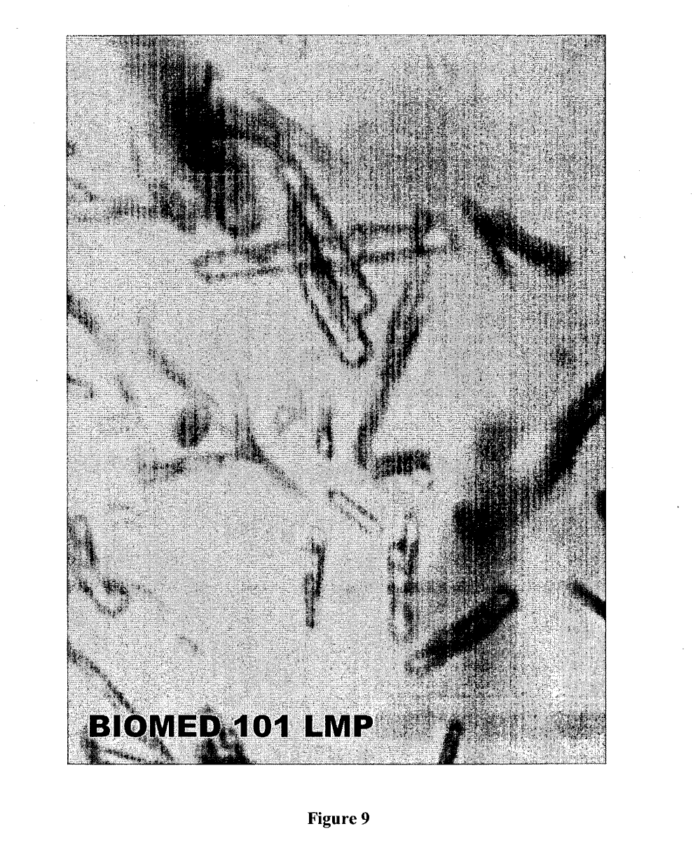

FIG. 10: This figure provides a photomicrograph of the previously unknown high melting point LBA of this invention. This is designated as BMED 101 HMP and has the same chemical name as shown in the description of FIG. 9.

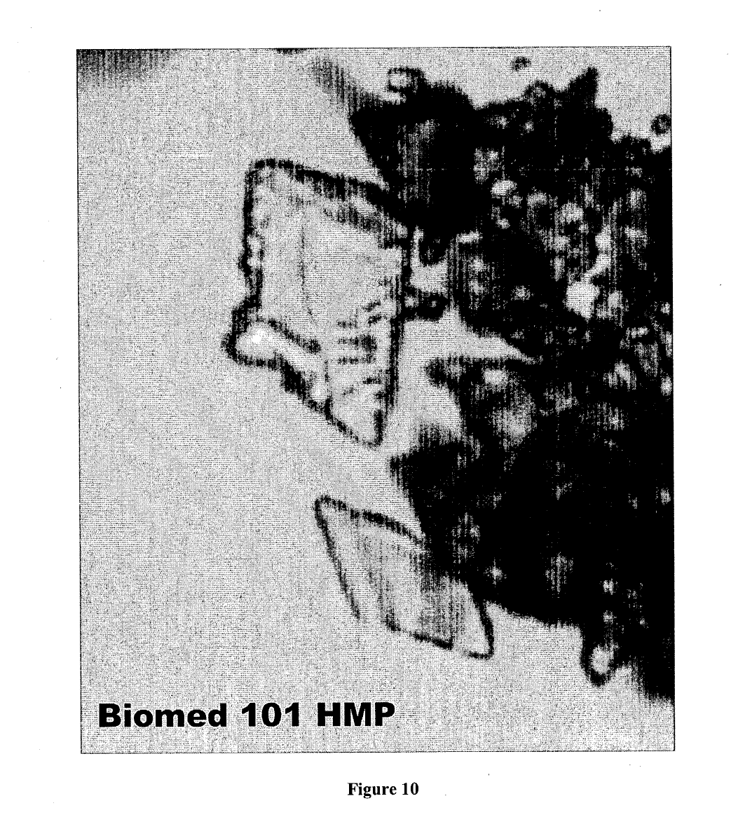

### DETAILED DESCRIPTION AND PRESENTLY PREFERRED EMBODIMENTS

For purposes of this application the following definitions apply:

LBA is the abbreviation for leukotriene B₄ antagonist, i.e. a compound that interferes with a leukotriene B₄ (LTB4) activity. Examples of LBAs may be found in U.S. Pat. No. 6,423,744B1, issued 23 Jul. 2002, which is incorporated in its entirety. The antagonist activity may be through inhibiting the synthesis of leukotriene B₄ or by interfering with the leukotriene B₄ receptor. Thus the LBA may be a LTB4 synthesis inhibitor or a LTB4 receptor antagonist, preferably the latter.

HMP means “high melting point” and is used as a convenient designator for the polymorphic form of this invention having a higher melting point than a different polymorphic form with a lower melting point.

LMP means “low melting point” and is also used as a convenient designator for the known polymorphic form having a lower melting point than the HMP polymorphic form of this invention.

A stereoisomer is one of a set of isomers whose molecules have the same atoms bonded to each other but differ in the way these atoms are arranged in space. Included in this are enantiomers, i.e., compounds that are mirror images of each other but that are not superimposable upon each other.

It should be understood that the use of the alternative “or” with items in a series is meant to include both the alternative and the collective. Thus, “preserving the antitumor, antiviral, or immunostimulatory effects” would include preserving each alone or in any combination.

The Compound of the Invention

The LBA of in this invention is one that blocks the effects mediated by the leukotriene B₄ receptor. An LBA may block the effects mediated by the LTB₄ receptor by acting directly on the receptor or by inhibiting the synthesis of LTB₄, preferably the former. The LBA of this invention is a compound represented by Formula (I) as follows:

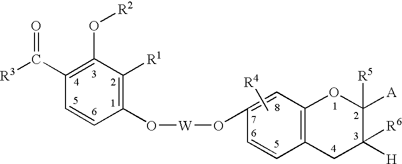

wherein

R¹ represents propyl;

R² represents methyl;

R³ represents methyl;

W represents (CH₂)ₓ where x is 3;

R⁴ represents propyl at the 8 position of the benzopyran ring;

R⁵ represents hydrogen;

R⁶ represents hydrogen; and

A represents COOH.

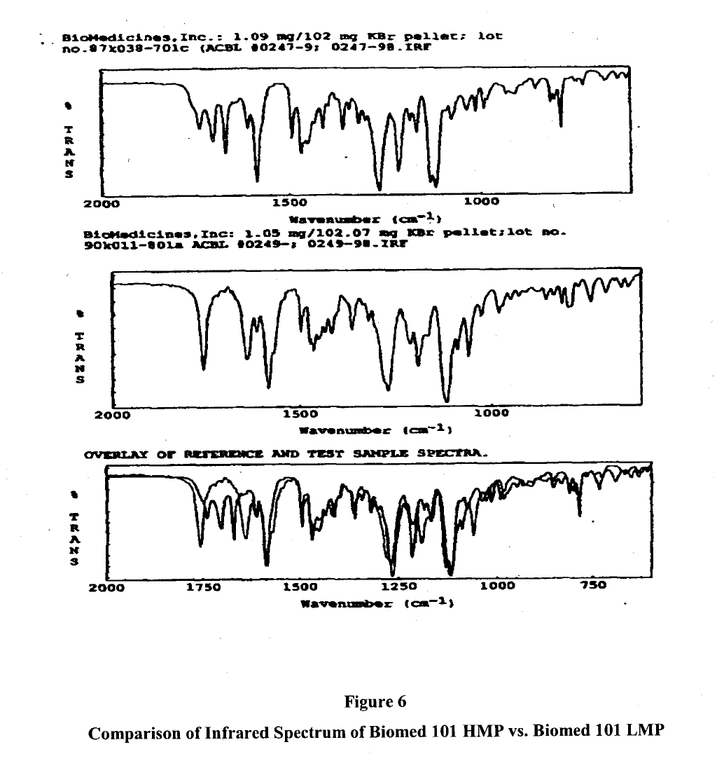

The name of the compound is (±) 7-[3-(4-acetyl-3-methoxy-2-propylphenoxy)propoxy]-3,4-dihydro-8-propyl-2H-1-benzopyran-2-carboxylic acid and is a racemic mixture of the two stereoisomers that exist due to the asymmetric carbon at the 2-position of the benzopyran ring. The compound has a melting point of about 80° C.-82° C. and is a hitherto unidentified polymorphic form of the compound. The compound exhibits unique characteristics discussed hereinafter and will be referred to as the HMP LBA at various points in the following description. The infra red spectrum of the HMP LBA differs from the infra red spectrum of the LMP LBA as seen in FIG. 6. The melting point of the compound of this invention is the temperature at which the solid state changes into the liquid state at standard atmospheric pressure, that is the temperature at which the highly ordered arrangement of particles changes to a more random arrangement that characterizes a liquid. The melting point may be determined by one of ordinary skill in the art using the various techniques available, from manual observation to automated equipment such as the Büchi® melting point/Range Apparatus such as Models B-540 or B-545. Inherent in the melting point determination is a slight variation that may be seen between individuals, their skill level, and the techniques used. Thus, while the compound of the invention is readily identified by its melting point of 80° C. to 82° C., the term “about” is employed to reflect the slight variation in the measurement that one of ordinary skill in the art would recognize as a result of different individuals using different standard techniques and equipment that are used in determining melting points in the pharmaceutical arts.

Preparation of the Compound Useful in this Invention

The known compound is prepared by processes set forth in U.S. Pat. No. 4,889,871 issued Dec. 26, 1989 to Djuric, et al. and U.S. Pat. No. 4,788,214 issued Nov. 29, 1988 to Cohen et al. These patents are incorporated herein by reference in their entirety. U.S. Pat. No. 4,665,203 issued May 12, 1987 discloses methods for making some of the intermediates used in making compounds of the present invention. The patent is also incorporated herein by reference.

The process as described above in prior patents produces a polymorphic form of the product of Formula (I) that melts in ranges of about 65-73° C. (65-68° C. in U.S. Pat. No. 4,889,871) (the low melting point polymorph or “LMPP”) while sequential recrystallization with ethyl acetate and hexane and vacuum drying reliably produces a form that melts at 80-82° C. (the HMP LBA) and exhibits a unique IR spectrum.

A further aspect of this invention is a process for preparing the compound described above. Broadly, the process comprises dissolving a starting material of (±)-7-[3-(4-acetyl-3-methoxy-2-propylphenoxy)propoxy]-3,4-dihydro-8-propyl-2H-1-benzopyran-2-carboxylic acid in ethyl acetate, cooling the resulting solution below about 10° C., adding hexane to the solution while mixing until a precipitate forms, separating the precipitate from the liquid, and drying the precipitated material.

To prepare the HMP LBA of this invention, the known LMPP alone (or in combination with the HMP LBA) is dissolved in solvent such as ethyl acetate, preferably under an inert atmosphere while mixing the two components. Generally the mixing is performed by stirring at about 100-200 revolutions per minute (rpm) using standard processing techniques at about ambient temperatures of about 10° C. to about 30° C. The inert atmosphere will be a non-reactive gas such as nitrogen, argon, and the like. Nitrogen is preferred. Enough ethyl acetate is used to dissolve the LBA. One will want to avoid using a great excess of the solvent, as the use of a large excess may make the ensuing precipitation step more difficult. For example, for a 30 gram (g) quantity of the LBA with about 100 milliliters (ml) of the solvent works well, although slightly more or less (e.g. 10%) solvent may be used. A particularly useful ratio is about 33.4 g of the LBA per 100 ml of solvent. Once a solution is formed, it is cooled to below ambient temperature, but above the temperature at which it would solidify, e.g. below about 10° C., preferably 6° C. Once the reduced temperature is reached, a liquid in which the LBA is less soluble is added to precipitate the HMP LBA. This liquid is preferably hexane, which is added slowly at the reduced temperature to induce precipitation of the desired HMP LBA. The hexane may be added in a single amount or in multiple amounts. To accelerate the precipitation process, seed crystals of a previously prepared HMP LBA may be added once the hexane has been mixed with the ethyl acetate LBA solution. The hexane is preferably added in two stages while the temperature and mixing, e.g. stirring, are maintained at a constant rate, e.g. about 100-200 rpm, preferably about 140 rpm. In the first stage, a volume that is about twice that of the ethyl acetate is added over a period of time that may vary from fifteen to thirty minutes, e.g. 24 minutes. In the second stage, a larger amount of hexane is added, e.g. about 1.5 to 2.5 times the amount of hexane is used in the first stage, preferably about 2 times the amount of hexane used in the first stage. A precipitate forms progressively, which is collected e.g. by filtration. The temperature of the precipitate is slowly raised to ambient, e.g. about 20° C., and the resulting precipitate is air dried for a short time, e.g. less than about 30 minutes, preferably no more than about 10 minutes, then dried under vacuum (e.g. 0.7 torr.) for less than about two hours, e.g. about one hour. The resulting material is then preferably ground to a fine powder (0.1-100 micron (μM) diameter, preferably 2-40 μM, most preferably 5-20 μM) dried under a vacuum (e.g. 0.7 torr.) for an extended period of time such as about 72 hours, then dried at an above ambient temperature, e.g. about 40° C., for about 24 hours or less, preferably 18 hours. This results in the HMP LBA having a melting point of about 80° C. to 82° C. and having the unique characteristics discussed hereinafter.

The dissolution and precipitation procedures can be repeated one or more times if desired. Generally the about same ratios of the amounts of solid to ethyl acetate to hexane will be used. The drying procedure can be repeated as well. Preferably, at least one recrystallization will be employed.

Administration to Treat Malignancies, Viral Conditions, and Immunological Diseases

Another aspect of this invention is a method for treating a malignancy, a viral condition, or immunological disease in a human subject having such malignancy, viral condition, or immunological disease. The method comprises administering to the subject a therapeutically-effective amount of IL-2 in conjunction with the HMP LBA described herein to reduce the adverse effects of IL-2. This method results in the level of IL-2 administered to the subject being greater than would be administered without the HMP LBA or that the length of time the IL-2 is administered is increased. Thus it can be said that the use of the HMP LBA of this invention improves the therapeutic index of IL-2 over what is known in the art, i.e. this invention improves the benefit-to-risk ratio.

Therapeutic index, in its most general form, is a benefit:risk ratio that relates the benefits derived from a particular treatment or therapy to the risks associated with that same treatment or therapy. Somewhat more mathematically, the therapeutic index may be calculated as the dose or dose level of a drug that provides useful clinical benefit as compared to the dose or dose level of the same drug that causes adverse events of such severity that the adverse-event causing dose is not tolerated. The ratio of these two doses or dose-levels has been described as the “therapeutic index.” Still a third useful definition is the ratio of the change in an objective benefit to the change in an objective risk caused by some type of intervention during therapy. The intervention could be the administration of another drug or drugs or the performance of a medical or surgical procedure, or a combination of these. Those skilled in the art will recognize that other definitions may exist which, nevertheless, connote the fundamental concepts described herein.

Most simply, a therapeutic index changes if the benefits change but the risks do not, or the risks change but the benefits do not. However, it is possible that risks and benefits may change simultaneously, in the same or opposite directions and with similar or different magnitudes. Then the direction and relative magnitude of the changes become determinant regarding the effect on the therapeutic index.

The possible changes in risks and benefits and the effect on therapeutic index are shown in Table 1 below.

TABLE 1
Effect of an Intervention on Benefits,
Risks, and Therapeutic Index

| Change in | | Therapeutic |
|---|---|---|
| Benefits | Change in Risks | Index |
| increase | no change | increase |
| increase | Decrease | increase |
| no change | Increase | decrease |
| decrease | no change | decrease |
| decrease | Increase | decrease |
| no change | Decrease | increase |
| increase | increase | increase |
| | (less than increase in | |
| | change in benefits) | |
| increase | increase | decrease |
| | (more than increase in | |
| | change in benefits) | |
| decrease | decrease | decrease |
| | (less than decrease in | |
| | change in benefits) | |
| decrease | decrease | increase |
| | (more than decrease in | |
| | change in benefits) | |

Note, however, that in order to determine the effect on therapeutic index of any intervention that effects either risks or benefits it is essential to determine the changes in direction and magnitude of both risks and benefits. No useful statement regarding the impact of an intervention on therapeutic index can be made without knowing simultaneously the effects on risks and benefits.

All medical judgments regarding the utility of a particular therapeutic, medical or surgical intervention are made on the basis of the therapeutic or interventional impact on the therapeutic index. This reliance on therapeutic index may be explicit or implicit but it is invariant in medical decision-making. We have found that the use of LBA HMP of this invention will improve the therapeutic ratio of IL-2 treatment by increasing the benefits of treatment while decreasing the risks.

It is known that human recombinant IL-2 is useful for treating certain malignancies, viral conditions, or other maladies. While human recombinant IL-2 is a well-studied, well-characterized and effective antineoplastic drug with well documented, often severe, and sometimes life-threatening or fatal side effects. One of the most serious side effects is VLS, which can affect the entire body and virtually every body system, organ, or tissue.

According to the “package insert” provided by Chiron Therapeutics, IL-2 (PROLEUKIN®) is a highly purified protein with a molecular weight of approximately 15,300 Daltons. The chemical name is des-alanyl-1, serine-125 human interleukin-2. IL-2, a lymphokine, is produced by recombinant DNA technology using a genetically engineered E. coli strain containing an analogue of the human interleukin-2 gene. Genetic engineering techniques were used to modify the human IL-2 gene, and the resulting expression clone encodes a modified human interleukin-2. This recombinant form differs from the native interleukin-2 in the follow ways: a) IL-2 is not glycosylated because it is derived from E. coli; b) the molecule has no N-terminal alanine; the codon for this amino acid was deleted during the genetic engineering procedure; c) the molecule has serine substituted for cysteine at amino acid position 125; this was accomplished by site specific manipulation during the genetic engineering procedure; and d) the aggregation state of PROLEUKIN® is likely to be different from that of native interleukin-2.

In addition, Chiron Therapeutics indicates that certain in vitro studies were performed to determine the properties of PROLEUKIN® and that these include: a) enhancement of lymphocyte mitogenesis and stimulation of long-term growth of human interleukin-2 dependent cell lines; b) enhancement of lymphocyte cytotoxicity; c) induction of killer cell (lymphokine-activated [LAK] and natural [NK] activity; and d) induction of interferon-gamma production. In in vivo studies, IL-2 produces multiple immunological effects in murine models in a dose-dependent manner. These include: a) activation of cellular immunity with profound lymphocytosis, eosinophilia, and thrombocytopenia; b) the production of other cytokines such as tumor necrosis factor, interleukin-1, and gamma interferon; c) inhibition of tumor growth. In addition, as noted previously, interleukin-2 has now been shown to stimulate the production of potentially toxic and inflammatory leukotriene B₄. Despite the large amount of knowledge concerning the effects of IL-2, the exact mechanism by which IL-2 mediates its antitumor (and toxic) effects in humans is unknown.

In general, the adverse pharmacological effect of IL-2 in a subject will occur during or after the treatment of the subject for an IL-2-responsive disease state. The method, along with other aspects of the invention, is useful in treating a subject having a leukotriene B4 receptor in its system. This generally includes mammals, such as livestock and pets, and particularly humans. Thus, this invention will find use in treating humans of all ages as well as in treating animals, i.e., in veterinary uses. The invention may be used for treating livestock such as cattle, sheep, pigs, goats, and the like or for treating household pets such as dogs, cats, rabbits, hamsters, mice, rats, and the like. The primary utility is for treating humans.

IL-2 is administered to a human as part of the treatment of a malignant tumor, i.e., cancer, or a viral disease such as AIDS, or an immunologic disease where the immune system of a patient is unbalanced or otherwise abnormal. Examples of the types of conditions treatable may be found in the most edition of The Merck Manual. The adverse pharmacological effect often seen in such treatment is increased vascular permeability, e.g., vascular leakage syndrome (VLS). The signs and symptoms of the adverse pharmacological effect are, for example, cardiovascular (hypotension requiring pressors; arrhythmias, pericardial effusion); pulmonary (congestion, dyspnea, pulmonary edema, hypoxemia); hepatic (increased bilirubin, jaundice, ascites); hematologic (anemia, thrombocytopenia, leukopenia); gastrointestinal (nausea, emesis, diarrhea, gastrointestinal bleeding); renal (oliguria/anuria, decreased excretory function); dermatologic (pruritus, erythema, rash); musculoskeletal (arthralgia, myalgia); neural (dysfunction of central or peripheral nervous system, epileptic seizures); general (fever, pain, fatigue, weakness, localized or generalized edema, infection, weight gain, headache). The method may be performed by administering the IL-2 and the LBA in combination as a unit dosage or the IL-2 and the LBA may be administered individually, with the LBA being administered before, during or after the administration of the IL-2. The HMP LBA of this invention is administered by a medically acceptable route of administration such as orally, parenterally (e.g., intramuscularly, intravenously, subcutaneously, intraperitoneally), transdermally, rectally, by inhalation and the like, preferably parenterally or orally before, during or after the IL-2 is administered.

Alternatively stated, another aspect of this invention is a method for reducing the adverse pharmacologic effects of IL-2 resulting from administration of IL-2 to treat a malignancy, a viral condition, or a immunological disease. The method comprises administering the HMP LBA of this invention in conjunction with the IL-2 at a level sufficient to reduce such adverse pharmacologic effects of the IL-2. The effective amounts and timing of HMP LBA administration are discussed hereinbefore and will result in an improved therapeutic ratio for the IL-2 treatment.

Another aspect of this invention is a method for enhancing the benefits of LBA treatment of a subject undergoing or preparing to undergo IL-2 treatment while at the same time reducing the adverse effects of LBA treatment. The method comprises administering an effective amount of the HMP LBA of this invention to the subject in conjunction with IL-2 instead of previously known LBA substances. This can be seen as improving the therapeutic index of the LBA treatment regimen.

An effective amount of HMP LBA will vary somewhat from subject to subject but generally will be in the range of about 0.1 mg to about 50 mg per kilogram of body weight per day. The preferred range is from 1 to 40 mg/kg/day while the most preferred range is from 3 to 25 mg/kg/day. Thus, for a 70 kg person, about 7 to 3500 mg/day would be administered, preferably, 70 to 2800 mg/day, most preferably 210 to 1750 mg/day of the HMP LBA of this invention. While these dosage ranges provide broad guidance to one of skill in the art for administering LBA to improve the therapeutic ration of IL-2 treatment, a range that is specific for an individual patient is preferably first established to maximize the benefit of treatment with a LBA, especially the HMP LBA of this invention.

Thus, another aspect of this invention is a method of determining a target range of a dosage of an LBA optimized for delivery to a specific human patient identified for treatment with IL-2 for a malignancy, a viral disease, or an immunologic disease, wherein the dosage of the LBA improves the therapeutic ratio of the IL-2. The method includes the careful evaluation of the patient's reaction to the IL-1 treatment alone, then in combination with the LBA. Initially the patient's reaction to IL-2 treatment alone is determined. This entails administering IL-2 in accordance with labelling instructions and evaluating the patients vital signs (for example pulse rate, systemic blood pressure, respiratory rate, core body temperature, and other factors discussed hereinbefore) to determine the patient's tolerance level. Next a LBA is administered to establish a patient plasma level of at least 1 μg/ml. The LBA dosage is increased while the IL-2 is maintained until the vital signs improve, whereupon the IL-2 is increased. The patient's vital signs are further monitored for adverse affects of IL-2 and the LBA. This process is continued until maximum benefit is seen for the combination treatment. Each patient will have a slightly different target dosage range at which the LBA can be administered and that target range may vary depending on the attending physicians evaluations. Once the optimum target range is established, the joint treatment with IL-2 and the LBA is continued until the attending physician determines an appropriate time to cease administration.

Still another aspect of this invention is an improvement of a previously known method of treatment. In a method for reducing the adverse effects of IL-2 in the treatment of a subject having a malignancy, a viral condition, or an immunological disease with IL-2 in conjunction with LBA, the improvement that comprises administering the HMP LBA instead of the previously used LBA.

Another aspect of this invention is not dependent on the use of the HMP LBA of this invention, but instead may employ any LBA, although the HMP LBA of this invention is preferred. This aspect is a method for treating a malignancy, viral condition, or immunologic disease in a subject having such a condition. The method comprises: (1) administering the LBA to the subject to maintain a level of the LBA in the subject's plasma within a target range; (2) thereby enabling the coadministration of IL-2 at a level greater than could be administered if IL-2 were given alone (so that the benefits of the higher level of IL-2 given with the LBA are greater than could be achieved with a lower level of IL-2 given without the LBA); (3) continuing to administer LBA and IL-2 to maintain the LBA within the range; and (4) optionally increasing the dosage of either or both the LBA and IL-2 if, after monitoring the subject's vital signs and laboratory parameters, such increase is warranted. For this aspect, any of the LBAs set forth in U.S. Pat. No. 6,423,744 B1, issued 23 Jul. 2002 may be used and the patent is incorporated herein by reference. The HMP LBA of this invention is preferred for use in this method, particularly where the plasma level of the LBA is maintained at about 1 μg/ml to about 20 μg/ml, preferably at about 2 μg/ml to about 16 μg/ml. The usual proposed IL-2 treatment regimen for a subject is to administer the indicated amount of IL-2 at least once a day (typically 3 times daily) for five consecutive days, then to cease the administration of IL-2 for approximately 9 days, and then to recommence administration of IL-2 at least once a day (typically three times daily) for the next five consecutive days. The LBA is administered to the subject prior to the IL-2 to establish a level of the LBA in the subject's plasma in the desired range. The LBA level is maintained for up to 24 hours, and most preferably for 6-12 hours, after the final IL-2 dose is given. The subject's vital signs (such as pulse rate, systemic blood pressure, respiratory rate, and core body temperature) and/or laboratory tests (such as blood oxygen level, renal function, cardiac function, etc.) are monitored to determine if the adverse effects of the IL-2 (e.g. VLS) are reduced. If so, then the level of IL-2 administered to the subject may be increased in an effort to derive more benefit from the IL-2 treatment without an overall increase in the incidence or severity of adverse effects from IL-2. Also, any adverse effects of the LBA administration are monitored, e.g. ALT level in the liver, and if such levels are low, the amount of the LBA is optionally increased. In the next round of treatment with IL-2 and the LBA, if appropriate, the levels are increased to accelerate the successful treatment of the primary condition, such as immunological, oncologic or viral disorders.

This may alternatively be viewed as an improvement in a method of treating a subject having a malignancy, a viral condition, or an immunological disease with IL-2 in conjunction with a LBA. The improvement comprises maintaining the subject's plasma level of the LBA within a target range. As before, any LBA is useful, e.g., the compound (±)-7-[3-(4-acetyl-3-methoxy-2-propylphenoxy)propoxy]-3,4-dihydro-8-propyl-2H-1-benzopyran-2-carboxylic acid, preferably the polymorphic form of the compound having a melting point of 80° C. to 82° C.

Pharmaceutical Compositions of the Invention

Another aspect of this invention is a pharmaceutical composition that comprises the HMP LBA of this invention in combination with a pharmaceutically acceptable excipient. The amount of active compound may vary from about 5% by weight to about 95% by weight, depending on the desired size of the composition. The remainder will be the excipient or excipients in amounts suitable for maintaining the integrity of the desired dosage form of the composition. Preferably the HMP LBA of this invention is first ground to a fine powder (about 0.1 to about 100 μM, preferably about 2-40 μM, and most preferably about 5-20 μM) before combining with the excipients.

Unit doses or multiple dose forms are contemplated, each offering advantages in certain clinical settings. The unit dose would contain a predetermined quantity of active compound calculated to produce the desired effect(s), for example, in the setting of IL-2 coadministration, e.g. a single tablet or capsule. The multiple dose form may be particularly useful when multiples of single doses, or fractional doses, are required to achieve the desired ends.

A unit dose will contain an amount of the HMP LBA of this invention sufficient to mitigate the adverse effects induced by excess leukotriene B₄ in a subject, but associated with an improved therapeutic index when compared to another polymorphic form of the LBA, and will contain an amount that will provide the desired dosage to the subject receiving the treatment. While the composition may be suitable for oral (enteral) or parenteral (intramuscular, intravenous, transdermal, intraperitoneal, subcutaneous) administration, the compound will preferably be administered orally. Suitable oral formulations include ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition. A tablet or capsule will contain about 25 to about 1200 mg of the HMP LBA, preferably about 50-500 mg, and most preferably about 200-400 mg.

The form may deliver the HMP LBA rapidly or may be a sustained-release preparation. The HMP LBA may be enclosed in a hard or soft capsule, may be compressed into tablets, or may be incorporated with beverages, food or otherwise into the diet. The percentage of the final composition and the preparations may, of course, be varied and may conveniently range between 5 and 95% of the weight of the final form, e.g., tablet. The amount of LBA in such therapeutically useful compositions is such that a suitable dosage will be obtained. Preferred compositions according to the current invention are prepared so that an oral dosage unit form contains between about 5 to about 50% by weight (% w) of the HMP LBA in dosage units weighing between 50 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the LBA, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

As pointed out above, the compound may be administered parenterally, e.g., intravenously, intramuscularly, intravenously, subcutaneously, or intraperitonieally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except in so far as any conventional media or agent is incompatible with the active ingredient, use in therapeutic compositions is contemplated. Moreover, other or supplementary active ingredients can also be incorporated into the final composition. The dosage of the parenteral dosage unit will be 0.1-100% of the oral dosage unit, preferably 10-100%, more preferably 30-100%, and most preferably 50-100%.

Solutions or suspensions of the LBA may be prepared in suitable aqueous or non-aqueous diluents such as water, glycol, ethanol, glycerol, polyethylene glycol, various oils, and/or mixtures thereof, and others known to those skilled in the art.

The pharmaceutical forms suitable for injectable use include sterile solutions or suspensions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered.

Alternatively, a slow long term infusion or multiple short term daily infusions may be utilized, typically lasting from 1 to 8 days. Alternate day or dosing once every several days may also be utilized.

Sterile, injectable solutions or suspensions are prepared by incorporating the compound in the required amount and, if necessary, of the required granularity into one or more appropriate solvents to which other ingredients, listed above or known to those skilled in the art, may be added as required. Sterile injectable solutions or suspensions are prepared by incorporating the compound in the required amount in the appropriate solvent with various other ingredients as required. Sterilizing procedures, such as filtration or irradiation, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases involving an injectable product the final form, as noted, must be sterile and must also be able to pass readily through an injection device such as a, hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized. Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

The following composition is representative for a capsule being about 100 mg total mass:

| | | |
|---|---|---|
| HMP LBA | 50 | mg |
| Lactose monohydrate, NF | 45-55 | mg |
| Hydroxypropylmethyl cellulose, USP | 1.8-2.2 | mg |
| Sodium lauryl sulfate, NF | 0.45-0.55 | mg. |

Article of Manufacture

Another aspect of this invention is an article of manufacture that comprises a pharmaceutical composition comprising the HMP LBA of this invention in a container associated with printed labeling instructions for administering the composition to a human subject having an IL-2 treatable malignancy, viral condition, or immunological disease in conjunction with the IL-2 to treat such malignancy, viral condition, or disease. Preferably the container holds a plurality of unit dosages, as discussed hereinbefore, and the amount of the composition administered is sufficient to reduce IL-induced adverse pharmacological effects in the subject being treated, as discussed above.

Having now described the various aspects of the invention, one can see that there are several advantages of this invention. The use of the HMP LBA of this invention enhances the effectiveness of leukotriene inhibition, reducing the duration, intensity, and/or cost of treatment with the LBA. Treatment regimens can be simplified. Laboratory testing and the frequency or intensity of clinical examinations can be reduced. Monitoring for adverse side effects can be performed less often without risk to the patient. Costs can be reduced.

There are also several specific advantages of this invention that flow from improving the therapeutic index of IL-2. It permits the administration of higher and more effective doses of IL-2 without increasing the risk of adverse effects from IL-2, especially VLS. Thus, the antitumor efficacy of the combined regimen (LBA+IL-2) is superior to IL-2 alone. It reduces or obviates the need to place patients into intensive care units and onto respirators in the case of severe pulmonary edema, or to place patients into cardiac or coronary care units in the case of severe arrhythmias or congestive heart failure or onto dialysis protocols in the case of renal compromise. It reduces intensive nursing care or supportive care or need of ICUs or CCUs. It reduces diagnostic testing required to monitor patient responses to IL-2 and to determine the success of therapeutic interventions required to mitigate IL-2-related adverse events. It reduces diagnostic testing needed to demonstrate that certain events are caused by IL-2 rather than by another agent. It reduces the costs associated with diagnosing or treating IL-2-induced adverse events, particularly those associated with VLS. It preserves or enhances the activity of IL-2 (or, at a minimum, reduces the adverse effects of IL-2 more than any reduction in activity of IL-2).

All patents, publications, and patent applications mentioned in this specification are herein incorporated by reference to the same extent as if each individual patent, publication, or patent application was specifically and individually indicated to be incorporated by reference.

### EXAMPLE 1

This example is a reproduction (without formulae) of Example 1 from U.S. Pat. No. 4,889,871 and sets forth a method for making the low melting compound 7-[3-(4-acetyl-3-methoxy-2-propylphenoxy)propoxy]-3,4-dihydro-8-propyl-2H-1-benzopyran-2-carboxylic acid.

(a) 493 mg of methyl 7-[3-(4-acetyl-3-hydroxy-2-propylphenoxy)propoxy]-3,4-dihydro-8-propyl-2H-1-benzopyran-2-carboxylate was added to 25 ml of acetone containing 276 mg of anhydrous potassium carbonate and 282 mg of methyl iodide. The mixture was refluxed for about 24 hours and water was added and the mixture was then extracted with ethyl acetate. The extract was dried, the solvent removed under vacuum, and the residual oil was chromatographed over silica gel with a 40/60 mixture of ethyl acetate/hexane to provide pure-methyl ether, methyl 7-[3[(4-acetyl-3-methoxy-2-propylphenoxy)propoxy]-3,4-dihydro-8-propy-2H-1-benzopyran-2-carboxylate.

(b) The methyl ether (340 mg) was dissolved in methanol (5 ml) containing lithium hydroxide (0.7 ml of a 2N LiOH solution, in water). The mixture was stirred at room temperature overnight and the solvent removed in vacuo. The residue was partitioned between ethyl acetate and 2N HCl and the organic layer separated and washed with brine. Evaporation of the volatiles in vacuo afforded crude acid. This material was purified by silica gel chromatography using ethyl acetate/hexane/acetic acid (40:60:0.5) as eluant. The pure product was recrystallized from ethyl acetate/hexane to afford 200 mg of product, 7-[3-(4-acetyl-3-methoxy-2-propylphenoxy)propoxy]-3,4-dihydro-8-propyl 1-2H-1-benzopyran-2-carboxylic acid, m.p. 65°-68° C.

Microanalysis: Found: C, 69.22; H, 7.53. Theory: C, 69.40; H, 7.49.

The NMR (CDCI₃) shows a —OCH₃ at δ3.75.

### EXAMPLE 2

This method teaches a process for preparing the HMP LBA of this invention.

A quantity of 20.7 g of material such as that from Example 1 is produced and dissolved in ethyl acetate (62 mL) at ambient temperature, under nitrogen, and while stirring at 140 rpm. The resulting solution is cooled to approximately 6° C. in an ice-water bath. Approximately 125 mL hexane is added to the solution over 24 minutes. At this point, it is useful to add a few seed crystals of the pure HMP, previously prepared, to aid in the precipitation. About 250 ml of hexane are next added over one hour, with constant temperature and continued stirring at 140 rpm. A precipitate forms progressively. A white solid is collected by filtration, brought slowly to 20° C., air-dried for ten minutes and then further dried under vacuum (0.7 torr) for one hour. The melting point of this solid is typically 80-82° C.

The dissolution process is repeated with the ethyl acetate (53 mL), hexane (105 mL), and seed crystals at the same conditions of temperature and stirring. The resulting solid was then warmed to room temperature, air-dried for ten minutes, and vacuum-dried to produce the second recrystallization solid.

The process was repeated with ethyl acetate (40 mL) and hexane (160 mL) and one-stage warming and two-stage drying as before.

The thrice-recrystallized solid is ground to a fine powder, dried under vacuum (0.7 torr) for 72 hours and then further dried at 40° C. for 18 hours.

The final 72- and 18-hour drying periods as described immediately above, may be used at the end of each crystallization cycle to obtain the HMP LBA of this invention.

### EXAMPLE 3

Dissolution of LMP and HMP Forms of the Compound of Formula I as a Function of pH

The LMP and HMP forms of the LBA of formula (I) were prepared in accordance with a process described in Examples 1 and 2, respectively. The pH of each of multiple 100 mL aliquots of distilled water was adjusted within the range of 1.2 to 7.5. Ten mg of either the LMP or HMP forms, finely ground with mortar and pestle, were placed into separate aliquots and then stirred at 37° C. for 15 mins. The dissolution characteristic of the LMP and HMP forms was measured at each pH and expressed as a percentage of the whole. Areas-under-the curve (AUCS) were calculated over the range of pH values tested. [Since the ordinate values are expressed as % dissolution and the abscissa values are expressed as pH units, then the units of AUC is expressed as the product of % dissolution times pH units or, alternatively, “%·log[H₃⁺]” Results appear below in Table 2.

TABLE 2

| Parameter | LMP | HMP |
|---|---|---|
| pH | Dissolution (%) | Dissolution (%) |
| 1.2 | 0 | 0 |
| 3.0 | 0 | 0 |
| 4.5 | 0 | 0 |
| 5.0 | 20 | 6 |
| 5.5 | 48 | 18 |
| 6.0 | 82 | 29 |
| 6.5 | 96 | 50 |
| 7.0 | 99 | 81 |
| 7.5 | 100 | 100 |

The AUC for the LMP over the range of pH was 284%·pH units vs 163%·pH units for the HMP. These data suggest although do not, by themselves, indicate that there may be any difference in the dissolution of the polymorphic forms as a function of pH. The data are consistent with the view that the HMP LBA form may remain in solid form somewhat more readily than the LMP compound during the transit of the bowel. These data are also consistent with the view that absorption may occur in the small bowel where pH is substantially more alkaline than in the stomach and proximal duodenum. However, somewhat more of the HMP is probably absorbed distally in the bowel than is the case for the LMP form.

### EXAMPLE 4

Pharmacokinetics of Suspension and Solution in the Rat

Adult male Munich-Wistar rats (250 g, Charles River Laboratories) received by gavage a suspension of either HMP or LMP LBA in polyethylene glycol 400 at 100 mg/kg or 100 mg/kg of an equal mass of the HMP and LMP forms in solution at pH 7.5. Non-compartmental pharmacokinetics were determined and appear below in Table 3.

TABLE 3

| | Cmax* | Tmax** | AUC*** |
|---|---|---|---|
| Form | (μg/mL) | (hrs) | (hr · μg/mL) |
| LMP | 10.4 | 0.7 | 57 |
| HMP | 13.0 | 2.0 | 65 |
| Solution | 1.0 | 8.0 | 8 |

*Cmax is defined as the maximum concentration in μg/mL of the compound found in the animals blood.
**Tmax is defined as the time required for the Cmax to be reached in the animal.
***AUC's were calculated by the trapezoidal rule.

These data suggest that a suspension of (solid) LMP or HMP is significantly better absorbed than a comparable mass of drug already completely dissolved. The data also suggest that the HMP form is absorbed somewhat more completely and somewhat more slowly that the LMP form.

### EXAMPLE 5

Effect of LMP LBA and HMP LBA on Hepatotoxicity in Humans

Human subjects between the ages of 18 and 70 received every 8 hours single capsules containing either the LMP LBA or the HMP LBA form. The drug is prepared as a fine powder and then mixed as drug/excipients at 20/80 wt %/wt % where excipients consist of equal weights of lactose and hydroxypropyl methylcellulose. The intention of dosing was to examine the effect of increasing doses of each LBA on safety and tolerability. Standard laboratory parameters were measured, including complete blood count, chemistry panel 20, urinary analysis, and electrocardiogram (ECG). Measurements of the laboratory tests were made daily except for the ECG which was measured at baseline, 1 week, and 1 week after cessation of dosing. There were no differences in the safety profiles with the exception of liver function testing.

Because the liver plays a central role in the metabolism of drugs, the effect a drug has on liver function is an important effect to consider. An indicator of an adverse effect that a drug may have on the liver is the peak change from baseline of alanine aminotransferase (ALT). Generally, if a drug is administered to a subject and the ALT increases significantly, the drug may be said to be hepatotoxic. A significant increase of ALT is generally an increase of more than 2. The ALT results are shown in Table 4.

TABLE 4

| | Cumulative | Peak Change from Baseline | |
|---|---|---|---|
| | Dose | Alanine Aminotransferase | |
| Daily | over 5 | (multiple) | |
| Dose (mg) | days (mg) | LMP | HMP |
| 60 | 300 | 2.5 | N/A* |
| 150 | 750 | 2.5 | N/A* |
| 75 | 375 | N/A* | 1 |
| 100 | 500 | N/A* | 1 |
| 125 | 625 | N/A* | 2 |
| 150 | 750 | N/A* | 2 |
| 175 | 875 | not dosed for reason of safety | 2 |
| 200 | 1000 | not dosed for reason of safety | 1 |
| 225 | 1125 | not dosed for reason of safety | 0 |
| 300 | 1500 | not dosed for reason of safety | −.5 |
| 450 | 2250 | not dosed for reason of safety | 0 |
| 600 | 3000 | not dosed for reason of safety | 2 |
| 750 | 3750 | not dosed for reason of safety | 1 |
| 900 | 4500 | not dosed for reason of safety | 1 |

*N/A = not applicable

In the case of the LMP LBA, 6/6 subjects were withdrawn from dosing at 60 and 150 mg/day because of the adverse changes in alanine aminotransferase levels. In contrast, and quite surprisingly, with the HMP LBA, significantly higher doses could be administered without excessive hepatotoxicity, i.e. doses up to 900 mg/day or 4500 mg over five days. We also observed that the LMP form administered daily for up to 14 consecutive days at 150 mg (total target dose=2100 mg) was not well tolerated by subjects because of hepatotoxicity. In contrast, with the HMP form and the use of intermittent dosing (5 days of HMP LBA, 9 days without exposure, followed by 5 days of HMP LBA again), the tolerability was improved even further despite the fact that the total exposure was 9000 mg LBA. Because of these unexpected and significant difference between the LMP LBA and the HMP LBA of this invention, the HMP LBA can be used at higher levels and in different regimens to provide the improved therapeutic ratio of the treatment of IL-2 responsive diseases.

We designate the HMP LBA as “hypotoxic.”

### EXAMPLE 6

The HMP LBA of this invention is formulated with the components set forth in Table 5.

TABLE 5
Components and Composition of Biomed
101 HMP 25 mg Capsules

| | | Representative |
|---|---|---|
| | Unit | Batch |
| Component | Formula* | Formula |
| HMP LBA | 25.0 mg | 100.0 g |
| Lactose, monohydrate NF | 225.05 mg | 900.2 g |
| Hydroxypropyl methylcellulose | 2.513 mg | 10.1 g |
| 2910, USP | | |
| Sodium lauryl sulfate, NF | 1.243 mg | 5.0 g |
| Purified water, USP | † | † |
| Hard gelatin capsule, opaque | 1 | 4000 |
| white - size #1 | | |

*Amounts may vary ±10% for components other than Biomed 101
† Used as a granulating agent and is removed during drying

### EXAMPLE 7

For 50 mg capsules, a more concentrated formulation was used that includes approximately 53 mg of excipients as shown in the table below. The approximate total weight of the 50 mg capsules is 103 mg not including the weight of the capsule shell.

TABLE 6
Components and Composition of Biomed
101 HMP 50 mg Capsules

| | | Representative |
|---|---|---|
| | Unit | Batch |
| Component | Formula* | Formula |
| HMP LBA | 50.00 mg | 200.00 g |
| Lactose, monohydrate NF | 50.00 mg | 200.00 g |
| Hydroxypropyl methylcellulose 2910, USP | 1.999 mg | 7.995 g |
| Sodium lauryl sulfate, NF | 0.502 mg | 2.009 g |
| Purified water, USP | † | † |
| Hard gelatin capsule - size #3 | 1 | 4000 |

*Amounts may vary ±10% for components other than Biomed 101
† Used as a granulating agent and is removed during drying

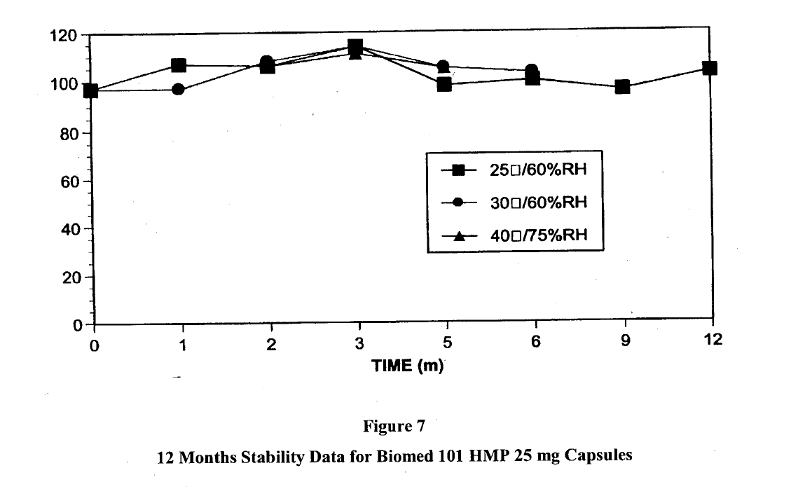

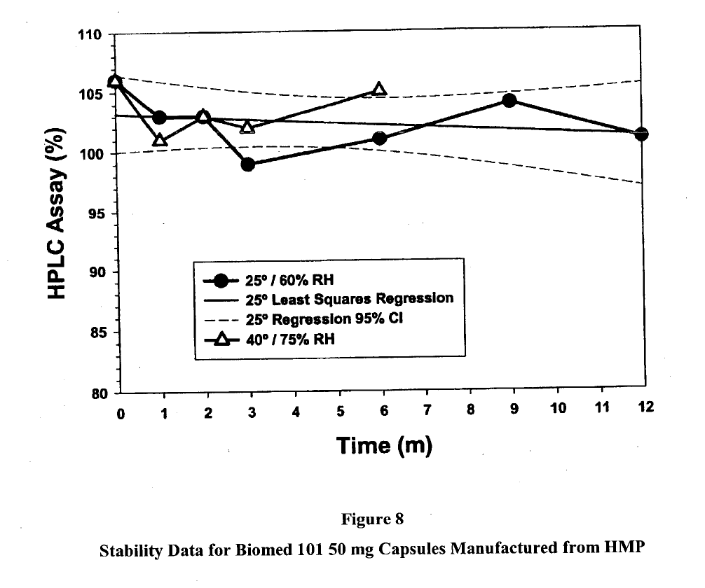

The above-described 50 mg capsules of the compositions of this invention were stability tested at 25° C. and 60% relative humidity (RH) and at 40° C. and 75% RH. The results of these stabilities tests are shown in tables 7A and 7B (25° C./60% RH) and 8A and 8B (40° C./75% RH). See FIGS. 7 and 8 for a graphical interpretation of some of the data.

TABLE 7A
Stability data - 25° C./60% RH

| Test/spec. | 0 release | 1 month | 2 month | 3 month |
|---|---|---|---|---|
| Appearance: | Conforms to | Conforms to | Conforms to | Conforms to |
| Sp: White opaque | specification | specification | specification | specification |
| capsules filled with | | | | |
| white to off-white | | | | |
| powder | | | | |
| Assay/HPLC | 106% | 103% | 103% | 99% |
| Sp: 90-110% | | | | |
| Dissolution: | | | | |
| Report results for | | | | |
| two points: | | | | |
| 20 min | 20 min 92% | 20 min 94% | 20 min 91% | 20 min 96% |
| 45 min | 40 min 98%* | 45 min 101% | 45 min 101% | 45 min 103% |
| | 50 min 99%* | | | |
| Disintegration | Meets specification | Meets specification | Meets specification | Meets specification |
| Sp: ≦15 min | (6 minutes 5 seconds) | | | |
| Impurities: | RRT 1.56 = 0.04% | RRT 1.4: 0.02% | RRT 1.4: 0.02% | RRT 1.4: 0.03% |
| Report results | | RRT 1.6: 0.03% | RRT 1.6: 0.04% | RRT 1.6: 0.04% |
| | | Total: 0.06% | Total: 0.06% | Total: 0.06% |
| Moisture | 2.4% | 2.4% | 2.3% | 2.1% |
| (Karl Fischer) | | | | |
| Microbial Limits: | Total aerobic microbial | Not tested | Not tested | Not tested |
| Report results | count: <100 CFU/g | | | |
| | Total yeast and mold | | | |
| | count: <100 CFU/g | | | |
| | Salmonella: none | | | |
| | E. coli: none | | | |
| | S. aureus: none | | | |
| | Ps. aeruginosa: none | | | |

*Dissolution testing was not performed at 45 minutes

TABLE 7B
Stability data - 25° C./60% RH

| Test/spec. | 6 month | 9 month | 12 month |
|---|---|---|---|
| Appearance: | Conforms to | Conforms to | Conforms to |
| Sp: White opaque capsules | specification | specification | specification |
| filled with white to off-white | | | |
| powder | | | |
| Assay/HPLC | 101% | 104% | 101% |
| Sp: 90-110% | | | |
| Dissolution: | | | |
| Report results for two | | | |
| points: | | | |
| 20 min | 20 min 97% | 20 min 90% | 20 min 102% |
| 45 min | 45 min 99% | 45 min 100% | 45 min 105% |
| Disintegration | Meets specification | Meets specification | Meets specification |
| Sp: ≦15 min | | | |
| Impurities: | RRT 1.4: 0.03% | RRT 1.4: 0.02% | RRT 1.4: 0.02% |
| Report results | RRT 1.6: 0.03% | RRT 1.6: 0.03% | RRT 1.6: 0.04% |
| | Total: 0.06% | Total: 0.05% | Total: 0.06% |
| Moisture | 2.1% | 2.3% | 2.9% |
| (Karl Fischer) | | | |
| Microbial Limits: | Total aerobic plate | Not tested | Total aerobic plate |
| Report results | count: | | count: |
| | <10 CFU/g | | <10 CFU/g |
| | Total yeast and mold | | Total yeast and mold |
| | count: <10 CFU/g | | count: <10 CFU/g |
| | Salmonella: none | | Salmonella: none |
| | S. Aureus: none | | S. Aureus: none |
| | Ps. Aeruginosa: none | | Ps. Aeruginosa: none |

TABLE 8A
Stability data - 40° C./75% RH

| Test/spec. | 0 release | 1 month | 2 month |
|---|---|---|---|
| Appearance: | Conforms to | Conforms to | Conforms to |
| Sp: White opaque capsules | specification | specification | specification |
| filled with white to off-white | | | |
| powder | | | |
| Assay/HPLC | 106% | 101% | 103% |
| Sp: 90-110% | | | |
| Dissolution: | | | |
| Report results for two | | | |
| points: | | | |
| 20 min | 20 min 92% | 20 min 91% | 20 min 94% |
| 45 min | 40 min 98%* | 45 min 98% | 45 min 103% |
| | 50 min 99%* | | |
| Disintegration | Meets specification | Meets specification | Meets specification |
| Sp: ≦15 min | (6 minutes 5 seconds) | | |
| Impurities: | RRT 1.56 = 0.04% | RRT 1.4: 0.02% | RRT 1.4: 0.02% |
| Report results | | RRT 1.6: 0.03% | RRT 1.6: 0.04% |
| | | Total: 0.06% | Total: 0.06% |
| Moisture | 2.4% | 2.5% | 2.0% |
| (Karl Fischer) | | | |
| Microbial Limits: | Total aerobic microbial | Not tested | Not tested |
| Report results | count: | | |
| | <100 CFU/g | | |
| | Total yeast and mold | | |
| | count: <100 CFU/g | | |
| | Salmonella: none | | |
| | S. Aureus: none | | |
| | Ps. Aeruginosa: none | | |

*Dissolution testing was not performed at 45 minutes

TABLE 8B
Stability data - 40° C./75%

| Test/spec. | 3 month | 6 month |
|---|---|---|
| Appearance: | Conforms to | Conforms to |
| Sp: White opaque | specification | specification |
| capsules filled with white | | |
| to off-white powder | | |
| Assay/HPLC | 102% | 105% |
| Sp: 90-110% | | |
| Dissolution: | | |
| Report results for two | | |
| points: | | |
| 20 min | 20 min 87% | 20 min 74% |
| 45 min | 45 min 100% | 45 min 97% |
| Disintegration | Meets specification | Meets specification |
| Sp: ≦15 min | | |
| Impurities: | RRT 1.4: 0.02% | RRT 1.4: 0.03% |
| Report results | RRT 1.6: 0.03% | RRT 1.6: 0.03% |
| | Total: 0.06% | Total: 0.06% |
| Moisture | 2.4% | 2.3% |
| (Karl Fischer) | | |
| Microbial Limits: | Not tested | Total aerobic plate count: |
| Report results | | <10 CFU/g |
| | | Total yeast and mold |
| | | count: <10 CFU/g |
| | | Salmonella: none |
| | | S. Aureus: none |
| | | Ps. Aeruginosa: none |

### EXAMPLE 8

A series of tests were run to determine the in vitro and in vivo pharmacology properties of the HMP LBA of this invention. A summary of the results of these tests appear below.

In Vitro Pharmacology

Inhibition of LTB4 binding to human neutrophils

IC50=0.3 micromolar

Inhibition of LTB4 chemotaxis

range=0.3-3.0 micromolar

Inhibition of human neutrophil adhesion to LTB4-stimulated umbilical vein endothelial cells

range=0.3-1.0 micromolar

Inhibition of LTB4-induced neutrophil granulation

range=1-3 micromolar

Inhibition of LTB4 synthesis

IC50=2.1 micromolar

Inhibition of LTA4 conversion into LTB4

IC50=20 micromolar

In Vivo Pharmacology

Inhibition of LTB₄ chemotaxis in guinea pigs

ED50=0.6 mg/kg i.g.

Inhibition of 12 (R)—HETE in guinea pigs

ED50=20 mg/kg i.g.

Inhibition of acetic acid colonic inflammation in rats and guinea pig

ED50=20 mg/kg i.g.

Inhibition of calcium ionophore dermal inflammation in the guinea pig ear

ED50=0.7 mg/ear

These in vitro and in vivo data establish the potency and selectivity of the preferred compound and are particularly relevant to diminishing, i.e. mitigating the unwanted effects of IL-2. These data are also particularly relevant to establishing that leukotriene B₄ mediated responses, including VLS, whether induced initially by administration of IL-2 or by other means, are blunted by the preferred compound. These data are consistent with the data seen for the known LMP LBA. For both the HMP LBA of this invention and the known LMP LBA, the pharmacological properties of the compounds are measured after the compounds are dissolved. While the pharmacological properties of the HMP LBA will not change relative to the LMP LBA, the extent and timing of the activity may differ based on the differences in absorption on bioavailability.

### EXAMPLE 9

This example explains how the HMP LBA compound of this invention is administered to humans to increase the number of antitumor doses of IL-2 that can be administered and well tolerated while preventing the increase in IL-2-induced adverse effects that are typically associated with increasing doses of IL-2.

Test Material

The HMP LBA compound (prepared in accordance with the process of Example 2) from drug substance lot # BA901 was supplied as 25 or 50 mg capsules Batch # 99G111 by BioMedicines, Inc. Each hard gelatin capsule contained either 25 or 50 mg HMP LBA plus excipients including lactose hydrous NF; hydroxypropylmethylcellulose 2910, 6 cps USP; sodium lauryl sulfate NF; purified water, USP; and sodium chloride.

Patients

Patients meeting the following criteria are eligible for treatment with the LBA and IL-2.

Men or women age 18 years or older

Pathologically confirmed renal cell carcinoma

Eastern Cooperative Oncology Group (ECOG) performance status 0 or 1 and predicted life expectancy of 12 weeks or more

For women, childbearing potential definitively terminated by surgery, radiation or menopause or child-bearing potential attenuated by use of an approved contraceptive method (IUD, oral contraceptive, or double-barrier device)

For women capable of becoming pregnant, negative serum beta-HCG pregnancy test within 7 days prior to initiation of Biomed 101 therapy

Patients meeting any of the following criteria are ineligible for treatment with LBA:

History of:

Significant neurological dysfunction including seizures, uncontrolled central nervous systemic metastases, or clinical signs of other significant neurological diseases

Active gastrointestinal bleeding

Signs of hepatic failure including encephalopathy

History of moderate or severe coronary artery disease (NYHA Class 3 or 4)

Renal insufficiency (serum creatinine>2.0 mg/dL)

Aspartate aminotransferase, alanine aminotransferase or serum bilirubin levels more than 2.5 times upper limit of normal

Hemoglobin<9 g/dL

A platelet count of less than 100,000 platelets per mm³

Protocol

PROLEUKIN® IL-2 is administered to the patient in accordance with the labeling instructions (in brief, 600,000 IU/kg every 8 hours as tolerated). The HMP LBA compound (prepared as in Example 2) is administered orally to the patient beginning eight hours prior to the first dose of IL-2, every eight hours thereafter during continuing IL-2 dosing, and once again eight hours after the final dose of IL-2. Typically, it is intended that IL-2 will be given three times daily for a total of 14 doses during a five-day period or course of treatment. Therefore, 16 doses of the LA compound would be given during this same five-day period. [In the event that there are IL-2 induced adverse side effects, treating physicians typically withhold the next one or more doses of IL-2. In such an instance, however, dosing with the HMP LBA compound continues.] Typically there is a nine-day rest period during which no IL-2 is given and then the second course of treatment is administered. These two courses constitute one cycle of IL-2. Additional cycles may be administered every two or three months dependent upon the response to and tolerability of the treatment by the patient.

Treating physicians normally prescribe the maximal tolerated dose of IL-2 in an effort to maximize the antitumor effect of IL-2. Accordingly, the number of doses of IL-2 that can be given constitutes the single best measure of the tolerability of IL-2. The response rate, as measured by tumor shrinkage or disappearance, is the measure of antitumor activity.

During the treatment period, patient and laboratory parameters are measured, including:

1. pulse and respiratory rate, temperature, blood pressure, body weight; results of general physical examinations

2. complete blood counts (hemoglobin, hematocrit, white blood cells and differential, platelets)

3. liver enzymes such as aspartate aminotransferase (AST) or alanine aminotransferase (ALT)

4. creatinine and blood urea nitrogen

5. and other tests such as blood gases, calcium, magnesium, albumin, total protein, bilirubin (total, direct, indirect), 5′-nucleotidase, alkaline phosphatase, cholesterol and the like.

In addition, IL-2 may cause of a number of serious side effects that require other tests or interventions, including computerized tomography, magnetic resonance imaging, ultrasound, x-rays, contrast enhancement, electrodcardiography, electroencephalography, aspiration of fluids from body cavities, biopsy of tissues or organs, and invasive procedures such as dialysis of the blood, the use of supplemental oxygen or mechanical ventilators, administration of pressor agents to maintain blood pressure, and the like. Accordingly adverse side effects are noted and interventions monitored and recorded.

Importantly, the number of doses of IL-2 are recorded, the dose level of the HMP LBA compound is recorded, and blood level of the HMP LBA compound is measured, and these parameters are related to one another and to other noteworthy events such as response to treatment or adverse side effects or both.

Results: In an open-label dose-escalation clinical trial in patients with metastatic renal carcinoma, IL-2 and the HMP LBA compound were co-administered to 62 subjects. The scheduled dosing regimen of IL-2 was 3× daily for a total of 14 doses over 5 days, followed by 9 days without IL-2, and then a repeat of the initial 14 doses over 5 days. The dose of IL-2 was 600,000 IU/kg administered intravenously every 8 hours. If the patient was not tolerating IL-2, a scheduled dose was withheld. IL-2 doses were not reduced. The dose of HMP LBA was administered orally one hour prior to each scheduled dose of IL-2 and again 8 hours after the final scheduled dose (for a total of 15 doses of HMP LBA during each 5-day IL-2 dosing period.) The dose of HMP LBA was continued whether or not the dose of IL-2 was administered or withheld. Both males and females of various ages were studied (Table 5).

TABLE 7

| Dose | Number | | | Median |
|---|---|---|---|---|
| (mg/day | of | | | Age |
| HMP) | Subjects | Male | Female | (years) |
| 75 | 15 | 10 | 5 | 61 |
| 100 | 3 | 2 | 1 | 65 |
| 125 | 3 | 3 | 0 | 55 |
| 150 | 8 | 6 | 2 | 52 |
| 175 | 3 | 2 | 1 | 51 |
| 200 | 3 | 3 | 0 | 60 |
| 250 | 3 | 3 | 0 | 59 |
| 300 | 3 | 2 | 1 | 59 |
| 350 | 3 | 1 | 2 | 52 |
| 400 | 3 | 3 | 0 | 60 |
| 450 | 3 | 2 | 1 | 55 |
| 500 | 3 | 2 | 1 | 52 |
| 600 | 3 | 3 | 0 | 54 |
| 750 | 3 | 2 | 1 | 66 |
| 900 | 3 | 2 | 1 | 60 |
| Total | 62 | 47 | 15 | 57 |

The HMP LBA compound was well tolerated and the maximal tolerated dose was not reached at 900 mg/day. The concentration of the LA in the plasma rose linearly in a dose-proportional manner (p<0.02).

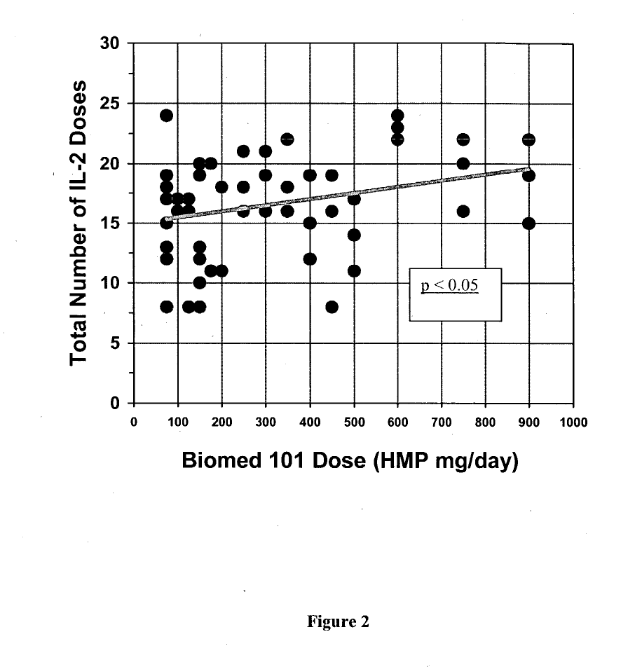

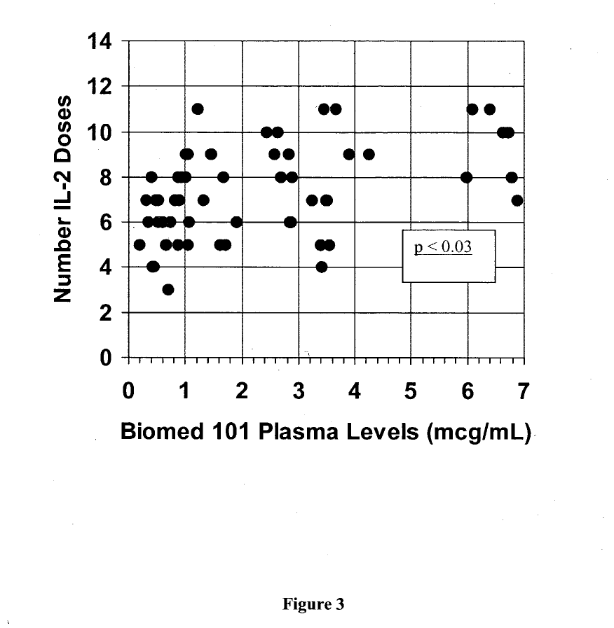

Because the HMP LBA is hypotoxic compared to the LMP, the dose and the plasma level of the HMP LBA could be substantially increased, and as a most important result, the number of doses of IL-2 that could be tolerated rose as well (FIG. 2, p<0.05 and FIG. 3, p<0.03, respectively). Plasma levels of the HMP LBA compound in excess of 1 μg/mL, and up to 10 μg/mL, were both well tolerated and effective in permitting a larger number of doses of IL-2 to be altered, with such IL-2 dosing being better tolerated with higher plasma levels of the HMP LBA.

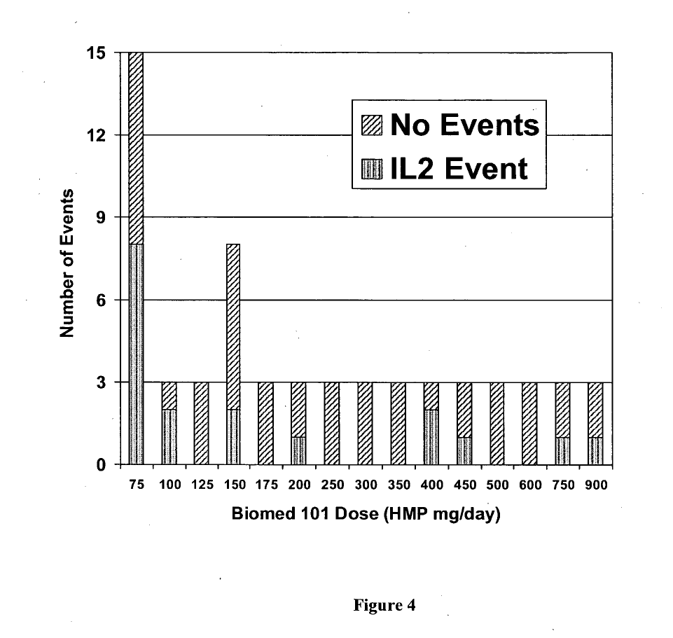

Typically, physicians will increase the dose of IL-2 to its maximally tolerated level in an effort to maximize clinical antitumor benefit. As a result, it is usually observed that the incidence and frequency of IL-2 induced serious adverse events increase sharply with the dose of IL-2. However, during coadministration of the HMP LBA of the current invention, the frequency of IL-2 induced serious adverse clinical events actually significantly declined as the total exposure to IL-2 increased (FIG. 4). At HMP LBA doses of <250 mg/day, the frequency of serious IL-2 events was 13 events observed in 35 patients (38%) while at doses at or above 250 mg/day, the frequency was reduced to only 5/27 (19%).

We have also observed that the response rate to treatment with IL-2 is not diminished by the prevention of IL-2 induced adverse events with the use of the LBA treatment. In patients with metastatic renal cell cancer, the objective response rate is 10-15% after a single course of treatment with IL-2. We expect these rates to be maintained or increased with further increases in IL-2 exposure achieved through the enhancement of safety permitted by the present invention.

Measurement of the HMP LBA plasma level and maintaining the HMP LBA level between about 1-20 μg/mL, preferably between 2-16 μg/mL, will improve the treatment of patients with cancer who are receiving IL-2. The dose of the HMP LBA should be changed as the patient condition or treatment changes in order to maintain the target plasma levels.

The invention now being fully described, it will be apparent to one of ordinary skill in the art that many changes and modifications can be made thereto without departing from the spirit or scope of the appended claims.

The subject matter claimed is:

1. A method for treating a malignancy, viral disease, or immunologic disease in a subject having such a condition, which method comprises
(a) administering a leukotriene B₄ antagonist (LBA) to the subject to maintain a level of the LBA in the subject's plasma within a target range,
(b) concurrently administering IL-2 at a level that is the same or greater than the dosage that can be tolerated by the subject for IL-2 administered in the absence of the LBA,
(c) continuing to administer the LBA and IL-2 to maintain the LBA within the individual target range, and
(d) optionally increasing the dosage of either or both the LBA and IL-2 if, after monitoring the subject's vital signs and/or laboratory parameters, such increase is warranted.

2. The method of claim 1, wherein the LBA is the compound (±)-7-[3-(4-acetyl-3-methoxy-2-propylphenoxy)propoxy]-3,4-dihydro-8-propyl-2H-1-benzopyran-2-carboxylic acid.

3. The method of claim 2, wherein the LBA is the polymorphic form of the compound having a melting point of about 80° C. to 82° C.

4. The method of claim 3, wherein the plasma level of the compound is maintained at about 1 μg/mL to about 20 μg/mL.

5. The method of claim 4, wherein the plasma level of the compound is maintained at about 2 μg/mL to about 16 μg/mL.

6. The method of claim 5, wherein the plasma level of the compound is maintained at about 3 μg/mL to about 9 μg/mL.

7. The method of claim 1, wherein the treatment cycle for administering IL-2 to the subject is administering the IL-2 for least once a day for 5 days, ceasing the administration of the IL-2 for the next 9 days, then resuming the administration of IL-2 for the next 5 days.

8. The method of claim 1, wherein the LBA is administered to the subject prior to administering IL-2 to establish a level of the LBA in the subject's plasma in the desired range.

9. The method of claim 8, wherein the LBA is administered after the final dose of IL-2 is administered so that the level of LBA in the patient's blood is maintained within the desired range.

10. The method of claim 1, wherein the adverse effects of IL-2 are monitored during treatment, and if the adverse effects are reduced, the amount of IL-2 administered to the subject is increased for the next treatment cycle, optionally with an increase of the plasma level of HMP LBA.

11. The method of claim 10, wherein the adverse effect is vascular leakage.

12. The method of claim 3, wherein, prior administering the LBA, the compound is reduced to a powder having a particle size in the range of about 0.1 μm to about 100 μm.

13. In a method of treating a subject having a malignancy, a viral disease, or an immunological disease with IL-2 in conjunction with a leukotriene B₄ antagonist (“LBA”), the improvement that comprises maintaining the subject's plasma level of the LBA within a target range during the IL-2 treatment of the malignancy or viral or immunological disease.

14. The method of claim 13, wherein the LBA administered to the subject is the compound (±)-7-[3-(4-acetyl-3-methoxy-2-propylphenoxy)propoxy]-3,4-dihydro-8-propyl-2H-1-benzopyran-2-carboxylic acid.

15. The method of claim 14, wherein the LBA is the polymorphic form of the compound having a melting point of about 80° C. to 82° C.

16. The method of claim 15, wherein the plasma level of the compound is maintained at about 1 μg/mL to about 20 μg/mL.

17. The method of claim 16, wherein the plasma level of the compound is maintained at about 1 μg/mL to about 16 μg/mL.

18. The method of claim 17, wherein the plasma level of the compound is maintained at about 2 μg/mL to about 16 μg/mL.

19. The method of claim 13, wherein the treatment schedule for administering IL-2 to the subject is administering the IL-2 for least once a day for 5 days, ceasing administration of the IL-2 for the next 9 days, then resuming administration of IL-2 for the next 5 days.

20. The method of claim 13, wherein the LBA is administered to the subject prior to administering IL-2 to establish a level of the LBA in the subject's plasma in the desired range.

21. The method of claim 20, wherein the LBA is administered after the final dose of IL-2 is administered so that the level of LBA in the patient's blood is maintained within the desired range.

22. The method of claim 13, wherein the LBA is administered intermittently.

23. The method of claim 22, wherein intermittent administration comprises dosing with the LBA beginning during the period from 72 hours before IL-2 dosing commences until 8 hours after IL-2 dosing commences and ending during the period from 24 hours before IL-2 dosing ends until two weeks after IL-2 dosing ends.

24. The method of claim 22, wherein intermittent administration comprises administering the LBA in two cycles of approximately equal duration, with an LBA-free period between the two LBA cycles of approximately 1-2 times the duration of the LBA administration period.

25. The method of claim 24, wherein the length of each LBA cycle is the same as the length of the attendant IL-2 cycle plus or minus one day.

26. The method of claim 22, wherein the total administered dose of the LBA given by intermittent dosing is greater than the dose of the same LBA that could be safely administered when given by continual or continuous administration.

27. The method of claim 24, in which the LBA cycle is 3-10 days.

28. The method of claim 13, wherein the adverse effects of IL-2 are monitored during treatment, and if the adverse effects are reduced, the amount of IL-2 administered to the subject is increased for the next treatment cycle, optionally with an increase in the plasma level of the HMP LBA.

29. The method of claim 28, wherein the adverse effect is vascular leakage.

30. The compound (±)-7-[3-(4-acetyl-3-methoxy-2-propylphenoxy)propoxy]-3,4-dihydro-8-propyl-2H-1-benzopyran-2-carboxylic acid, which exhibits a melting point of about 80° C.-82° C.

31. The compound of claim 30 in the form of a powder having a particle size in the range of about 0.1 μm to about 100 μm.

32. A method for mitigating the adverse effects of the administration of IL-2 to a human undergoing IL-2 treatment for a malignancy, a viral disease, or immunological disease, which method comprises administering the compound of claim 30 to the human in an amount and for a time sufficient to improve the therapeutic ratio of the IL-2.

33. A pharmaceutical composition that comprises a compound of claim 30 in combination with a pharmaceutically acceptable excipient.

34. The composition of claim 31, wherein the composition is a unit dosage form comprising about 50 mg to about 500 mg of the compound of claim 30.

35. The composition of claim 34, wherein the dosage form comprises about 200 mg to about 400 mg of the compound.

36. The composition of claim 35, wherein the compound is in the form of a powder of a particle size of about 0.1 μm to about 100 μm.

37. An article of manufacture that comprises the pharmaceutical composition of claim 30 in a container associated with printed instructions for administering the pharmaceutical composition to a human subject having an IL-2 treatable malignancy, viral disease, or immunological disease in conjunction with IL-2 to treat such malignancy or disease.

38. The article of claim 37, wherein the instructions describe a method that comprises
(a) administering the pharmaceutical composition to the subject to maintain a level of the compound in the subject's plasma within a fixed range,
(b) concurrently co-administering IL-2 at a level greater than the dosage recommended for IL-2 alone,
(c) continuing to administer the pharmaceutical composition and IL-2 to maintain the compound within the range, and
(d) optionally increasing the dosage of both the pharmaceutical composition and IL-2 if, after monitoring the subject's vital signs, such increase is warranted.

39. The article of claim 38, wherein the instructions indicate that the plasma level of the compound is maintained at about 1 μg/mL to about 20 μg/mL.

40. The article of claim 38, wherein the instructions indicate that the plasma level of the compound is maintained at about 2 μg/mL to about 16 μg/mL.

41. The article of claim 5, wherein the instructions indicate that plasma level of the compound is maintained at about 3 μg/mL to about 9 μg/mL.

42. The article of claim 38, wherein the instructions for administering IL-2 to the subject indicate administering the IL-2 for least once a day for 5 days, ceasing administration of the IL-2 for the next 9 days, then resuming administration of IL-2 for the next 5 days.

43. The article of claim 38, wherein the instructions indicate that the pharmaceutical composition is administered to the subject prior to administering IL-2 to establish a level of the compound in the subject's plasma in the desired range.

44. The article of claim 43, wherein the instructions indicate that the pharmaceutical composition is administered after the final dose of IL-2 is administered so that the level of the compound in the patient's blood is maintained within the desired range.

45. A process for preparing a pharmaceutical composition, which process comprises combining the compound of claim 30 with a pharmaceutically acceptable excipient.

46. The process of claim 45, wherein the compound is in the form of a powder having a particle size between about 0.1 μm to about 100 μm.

47. The process of claim 45, which further comprises preparing a unit dosage form with the composition.

48. A process for preparing the compound of claim 30, which process comprises
dissolving a starting material of (±)7-[3-(4-acetyl-3-methoxy-2-propylphenoxy)propoxy]-3,4-dihydro-8-propyl-2H-1-benzopyran-2-carboxylic acid in ethyl acetate,
cooling the resulting solution below 10° C.,
adding hexane to the solution while mixing until a precipitate forms,
separating the precipitate from the liquid, and
drying the precipitated material.

49. The process of claim 48, wherein seed crystals of previously formed compound of claim 30 are added to the mixture of the starting material, ethyl acetate, and hexane.

50. The process of claim 49, wherein the precipitated material is dried for up to 72 hours between about 20° C. and about 40° C.

51. The process of claim 48, wherein the dried precipitated material is dissolved in ethyl acetate, the solution cooled below about 10° C. and mixed with hexane until a precipitate forms, and the resulting precipitate is separated from the liquid and dried.

52. The process of claim 51, wherein the steps of claim 48 are repeated.

53. A method for assaying the effectiveness of treatment of a patient having a malignancy, a viral disease, or immunological disease, with IL-2 in conjunction with a leukotriene B₄ antagonist (“LBA”), which method comprises monitoring the patient's plasma levels for the LBA to determine the concentration of the LBA and adjusting the amount of the LBA administered to the patient to ensure the LBA concentration is maintained in a fixed range.

54. The method of claim 53, wherein the LBA is the compound (±)-7-[3-(4-acetyl-3-methoxy-2-propylphenoxy)propoxy]-3,4-dihydro-8-propyl-2H-1-benzopyran-2-carboxylic acid.

55. The method of claim 54, wherein the LBA is the polymorphic form of the compound having a melting point of about 80° C. to 82° C.

56. The method of claim 55, wherein the plasma level of the compound is maintained at about 1 μg/ml to about 20 μg/mL.

57. The method of claim 46, wherein the plasma level of the compound is maintained at about 2 μg/mL to about 16 μg/mL.

58. The method of claim 57, wherein the plasma level of the compound is maintained at about 3 μg/mL to about 9 μg/mL.

59. A method for mitigating leukotriene B4 receptor agonist (“LBA”) related adverse events in a process for treating a malignancy, a viral disease, or immunological disease using IL-2 in combination with the LBA (±)-7-[3-(4-acetyl-3-methoxy-2-propylphenoxy)propoxy]3,4-dihydro-8-propyl-2H-1-benzopyran-2-carboxylic acid, which method comprises using the specific polymorphic form of the LBA a melting point of about 80° C. to 82° C.

60. A method for mitigating the adverse effects of the administration of IL-2 to a human undergoing IL-2 treatment for a malignancy, a viral disease, or immunological disease, which method comprises administering the compound of claim 30 to the human in an amount and for a time sufficient to improve the therapeutic ratio of the IL-2.
